(19) United States
(12) Patent Application Publication
(10) Pub. No.: US 2023/0034055 A1
(43) Pub. Date: Feb. 2, 2023

(54) Monitoring Of Physiological Data In Animals

(71) Applicant: Rex Sense LLC, Oakland Township, MI (US)

(72) Inventors: Travis PETERSON, Oakland Township, MI (US); Andrew SNIDER, Oakland Township, MI (US)

(21) Appl. No.: 17/725,609

(22) Filed: Apr. 21, 2022

Related U.S. Application Data
(60) Provisional application No. 63/227,396, filed on Jul. 30, 2021.

(51) Int. Cl. A61B 5/0205; A61B 5/08; A61B 5/11; G16H 40/67; A61B 5/00

(52) U.S. Cl. CPC A61B 5/0205; A61B 5/0816; A61B 5/1118; G16H 40/67; A61B 5/0022; G16H 40/63

(57) ABSTRACT

A system for noninvasively monitoring physiological data of non-human animals, said system includes a sensor module having a transmit antenna, a receive antenna and a microprocessor. The transmit antenna being configured to wirelessly transmit electromagnetic waves to an area in the vicinity of an animal. The receive antenna being configured to receive modulated signals back from the animal that have been modulated as a function of physiological characteristics of animal. A hub having a processor and configured to receive signals from the sensor module. The processor in the hub being further configured to compare the signals from the sensor module with previously obtained data and generate an alert to a user if the comparison indicates an abnormal physiological characteristic of the animal.

### BACKGROUND OF THE INVENTION

### Technical Field

Embodiments of this invention relate to the non-invasive, non-contact monitoring of physiologic data in non-human animals in various states of restraint and confinement including communication methods, interpretations, accessibility, and management of relevant data.

### Discussion

Accurate, non-contact monitoring of physiological indicators (motion/sleep data, respiratory rate, heart rate, movement, and others) can benefit the veterinary industry by limiting stress-induced variability in data, improving animal safety by limiting direct access to monitoring equipment and enabling continuous physiologic monitoring in unsupervised scenarios.

Specifically, current solutions require physical contact with the animal being monitored—either via wired sensors, wearable monitors, or surgical implanted monitoring devices. Limitations of these solutions include patients refractory to wearing monitors, inaccuracy of physiologic data due to stress associated with monitoring devices, and risk factors associated with animals coming in contact with electronics. Such risk factors could be animals becoming further stressed by added components & impacting their health negatively, an animal being tangled in wires and causing severe harm or death, or any form of animal interaction causing false readings to a device through shifting of placement, detachment, or otherwise, with no continuous human observation.

Moreover, wireless monitoring would enable the collection of valuable psychologic data in clinical scenarios where current solutions add too great of risk to be safely implemented, such as patients inside a supplemental oxygen chamber. Currently no added wires or such devices may be permitted inside oxygen chambers due to potential catastrophic health hazards. A wireless such device placed in proximity to the animal outside the parameters of the wall or even within can create a much-needed continuous monitoring situation for critically ill animals. Currently a veterinary professional or technician is required to physically monitor such animals and expend crucial time & effort in checking & re-checking the animals and in rare cases missing warning signs of impending crashes or emergency situations. A wireless solution could provide immediate, real time indication of a crash or rapid deterioration in health currently unavailable with existing methods. Further end uses could be identified such as environmentally controlled animal treatment or housing cages, barometric chambers, shipping containers, or animal transportation crates among many others.

An animal in transit on plane, road, or sea does not receive constant monitoring, particularly in a stressful scenario. Recent examples of animals perishing in commercial flights show the risk to an animal in transit, for example, by air. Such wireless monitoring devices would provide commercial viability to allow for continuous monitoring of animals in transit. Such use cases could be domestic or other pets being transported by air via main cabin, cargo, or other means. Further cases could be equine or other animals being transported by sea, air or road where a continuous human presence either is not feasible or viable.

Integration or utilization during imaging (e.g. radiograph, CT, or MRI) or other diagnostic procedures would enable vitals monitoring concurrent to use of such devices that cannot allow for metal or other type materials either directly within or within close proximity to otherwise sensitive equipment. Even currently available wearable biometrics devices enabled with wireless data transmission are of limited use due to their inclusion of sensor equipment, often requiring metal or otherwise undesirable components, in close proximity to the animal and diagnostic equipment being utilized. A wireless, non-contact device using EM waves could provide a continuous monitoring solution for such applications where existing technologies are unable to be safely implemented.

Overnight veterinary observation including runs, cages, or other enclosures currently rely on continuous checking by human professionals. Such current monitoring methods are not utilized without a human present due to the inherent risks within those monitoring solutions. A wireless monitoring method would provide continuous monitoring without a veterinary professional needing to be present, thereby increasing the likelihood of receiving real time alerts to an animal in distress requiring immediate attention and otherwise recording physiologic data with a higher degree of efficacy and efficiency than is currently available.

Veterinary practice, both medical and surgical, would benefit from readily available physiological monitoring of unrestrained animals. Such monitoring would also enable more precise and accurate animal evaluation and training. Such monitoring can also be beneficial to ecological or behavioral studies of free ranging animals. Emergency situations where time is of the essence may not offer the ability to get animals hooked up to monitors, particularly when in distress and extremely volatile. Such a situation would benefit from wireless monitoring able to be deployed non-invasively.

Integration or utilization during diagnostic imaging procedures (e.g. radiograph, CT, or MRI) in which traditional monitoring equipment may be of limited utility. Such devices cannot allow for metal or other type materials either directly within or within close proximity of such device. Wearables fall into the same category and would not be capable of being present, in fact, causing major issue should they be attempted to be used in such a setting. A wireless such device utilizing a variety of EM waves could provide a continuous monitoring solution for such applications currently without capabilities using existing technology.

Wireless physiologic monitoring could also be used in zoo and wildlife conservation scenarios, such as during anesthetic recovery, where vitals are at some point limited by the inability to attach available monitoring solutions to the patient, either prior to achieving sedation and/or during anesthetic recovery. The ability to monitor respiratory rate and reduction in overall motion, for example, would provide valuable insight into the level of sedation prior to human interaction with the patient. Additionally, presence detection via deployment of these wireless monitors through complex animal housing enclosures would enable screening to ensure enclosures are empty prior to staff entering an area that they could encounter an unconfined animal.

Consumer facing benefits also exist with the opportunity to offer, via an app, access to the data (and possible camera & microphone) to view animal condition live while an animal is housed elsewhere. This could also allow for veterinarians and other professionals to collect meaningful data from home or other environments for interpretation avoiding the “white coat effect”.

### SUMMARY

According to an aspect of this invention single, or multiple, sensor(s) that utilize wireless sensing, preferably electromagnetic (EM) wave based sensing, to monitor animal movement, vital signs including heart rate & respiratory rate, agitation, stress levels, and other specific attributes that may or can change constantly. This device may be integrated into a cage door, wall, base, or top. Additionally, one embodiment may be a free-standing device which may be affixed to any surface including a cage door, wall, or other area to monitor an animal non-invasively. The data will be collected via the sensor and communicated back to a hub or device directly via wire, Bluetooth, WiFi, or other means. Provision is made to interpret such data and may or may not utilize algorithms, AI, or machine learning to achieve a visual representation of the data. This data will be expressed in a visual manner either in a computer device or wireless device such as a phone or tablet.

In a preferred embodiment, a programmable computer is programmed with software that may utilize any variety of machine learning or artificial intelligence to recognize patterns or interpret data further. Over time such software may build a database allowing for quicker interpretation of such data as well as increased accuracy. Such software may allow for inputs such as breed, animal size or classification (e.g. large dog, small dog, cat, horse, etc.).

One embodiment may also integrate a camera and/or microphone into the aforementioned device to provide additional functionality. Such end application may offer the end consumer the ability to monitor animals via their vital signs or visually. This camera may also be utilized to provide additional movement or meaningful data to the software to apply analysis and interpret further.

Another embodiment may deploy the application into an existing architecture such as a door to a cage, enclosure, barometric/oxygen chamber or any number of end uses. Such use would allow integration into additional products and avoid complexity of secondary devices being managed.

Another embodiment may deploy the application as a standalone sensor to be placed in proximity to the animal subject. Such use would allow deployment in dynamic environments or other scenarios where a field monitoring solution may be utilized.

### BRIEF DESCRIPTION OF THE DRAWINGS

The drawings described herein are for illustrative purposes only of selected embodiments and not all possible implementations, and are not intended to limit the scope of the present disclosure.

FIG. 1 shows an overview illustration of a multi-device deployment scenario.

FIG. 2 shows a schematic diagram of one embodiment of the sensor module shown in FIG. 1.

FIG. 3 shows a schematic diagram of one embodiment of the hub module shown in FIG. 1.

FIG. 4 shows a block diagram related to sensor module functions.

FIG. 5 shows a block diagram related to hub module functions, including data pooling, refinement and transmission.

FIG. 6 shows sensor placement options for a specific subject.

FIG. 7 shows potential mounting options to aid in sensor placement.

FIG. 8 shows a schematic diagram of the mobile monitor unit.

FIG. 9 shows a block diagram related to the function of the mobile monitor unit in FIG. 8.

FIG. 10 shows an illustration of a possible embodiment of the sensor module in FIGS. 1-2.

FIG. 11 shows an illustration of a possible embodiment of the hub module in FIGS. 1, 3.

FIG. 12 shows an illustration of a possible embodiment of a mobile monitor unit for use with sensor and/or hub modules in FIGS. 1-3.

FIG. 13 shows an illustration of a possible embodiment of the hub module with a detachable mobile monitor unit.

FIG. 14 shows an illustration of a stationary monitoring system or other computer not directly integrated into sensor or hub modules.

FIG. 15 shows an illustration of data sampling using EM sensing of an animal subject, via the sensor module described in FIG. 2.

FIG. 16 shows a block diagram of EM transmission and data sampling for a sensor module.

FIG. 17 (a) shows a flow chart describing how the interaction of transmitted EM waves with a surrounding environment produces measurable changes or deviations in EM waves that can be used to gather information on physical matter inside an area of detection.

FIG. 17 (b) shows a flow chart illustrating how the measurable changes to transmitted EM waves can sampled and converted into an electric signal that can subsequently be used to assess physiologic data in a non-human animal.

FIG. 18 shows a flow diagram for physiologic data monitoring and/or decision making process according to one embodiment.

FIG. 19 shows a flow diagram for implementation in patients with anomalous or otherwise actionable data reading.

FIG. 20 shows an example graphic depiction of sleep as the monitored physiological indicator.

FIG. 21 shows an example graphic depiction of respiratory rate as the monitored physiological indicator.

FIG. 22 shows an example graphic depiction of heart rate as the monitored physiological indicator.

FIG. 23 is a visual representation of a recorded phase shift obtained during patient monitoring in which both respiratory and heart rate are discernible from the same data set.

FIG. 24 is a flow chart describing the use of EM sensing to detect [various perimeters of vs. location and motion in] a non-human animal and how that data can be analyzed over time to monitor for physiologic endpoints.

FIG. 25 is a block diagram outlining user permissions and account access to the database.

FIG. 26 is a flow diagram detailing database handling of sensor module and user data inputs.

FIG. 27 shows an illustration of a web or app-based user interface to display collected data, alerts, or notifications.

### DETAILED DESCRIPTION OF THE PREFERRED EMBODIMENTS

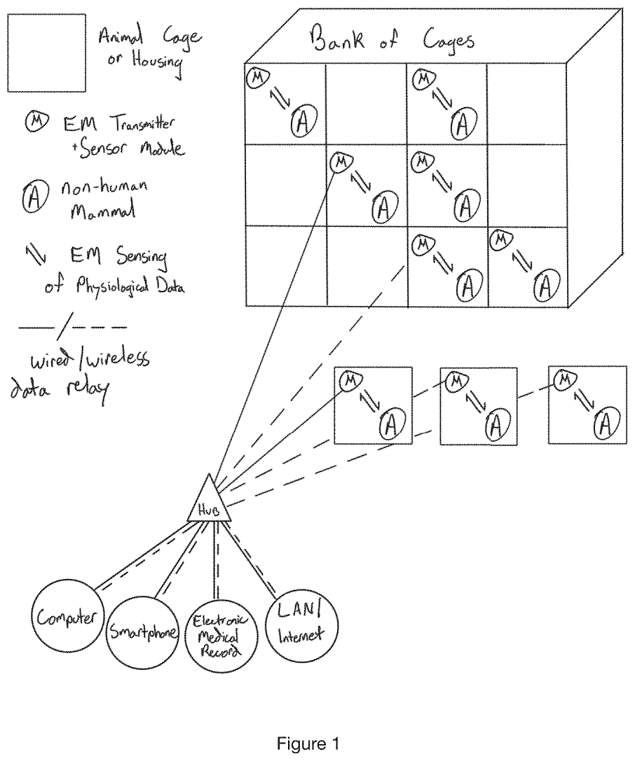

FIG. 1 shows an overview of a deployment scenario that uses multiple sensors M in close proximity to one another. With each sensor, they will individually transmit information to a central hub via direct-connection, WiFi, or Bluetooth. Such sensor will additionally draw power via a direct connection from the hub or outlet as well as optional battery method as referenced herein. The hub will take the details and communicate to any output device (tablet, phone, computer, database, etc.) or medical record system/database to log and view the data live or at a later date.

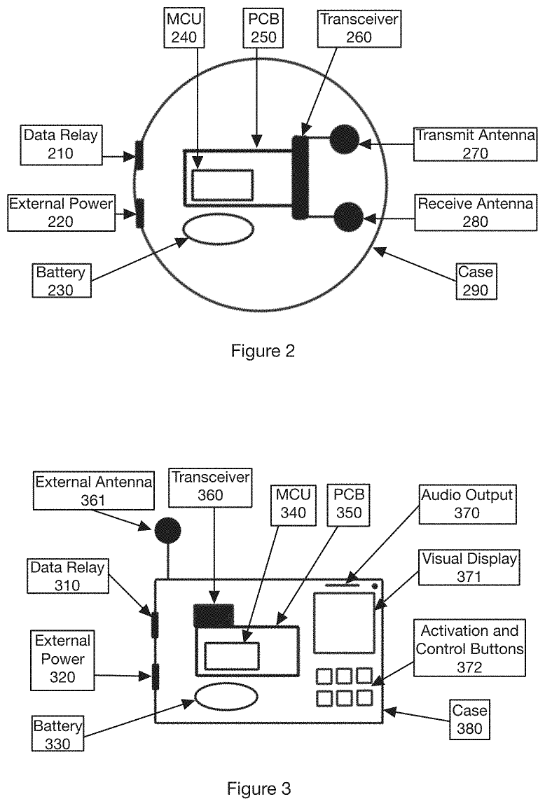

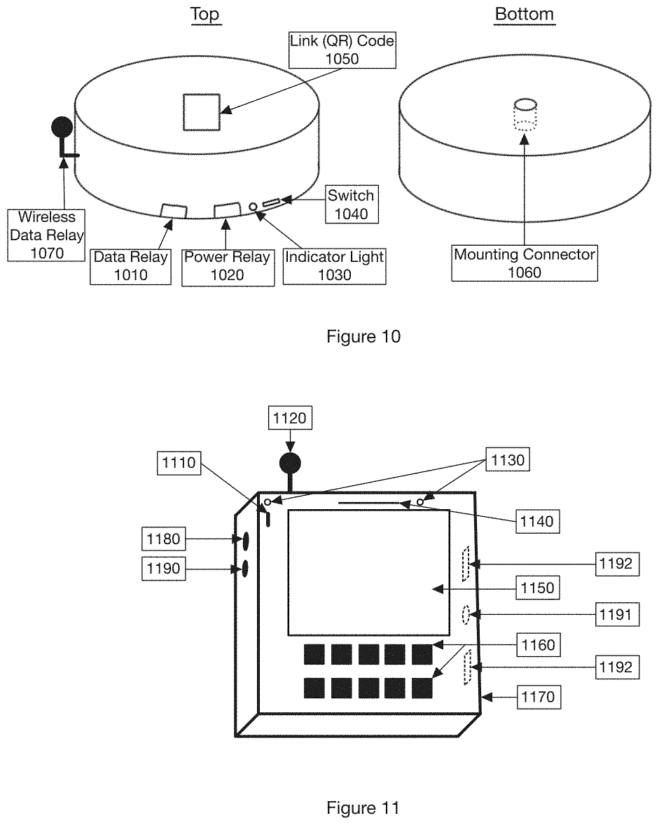

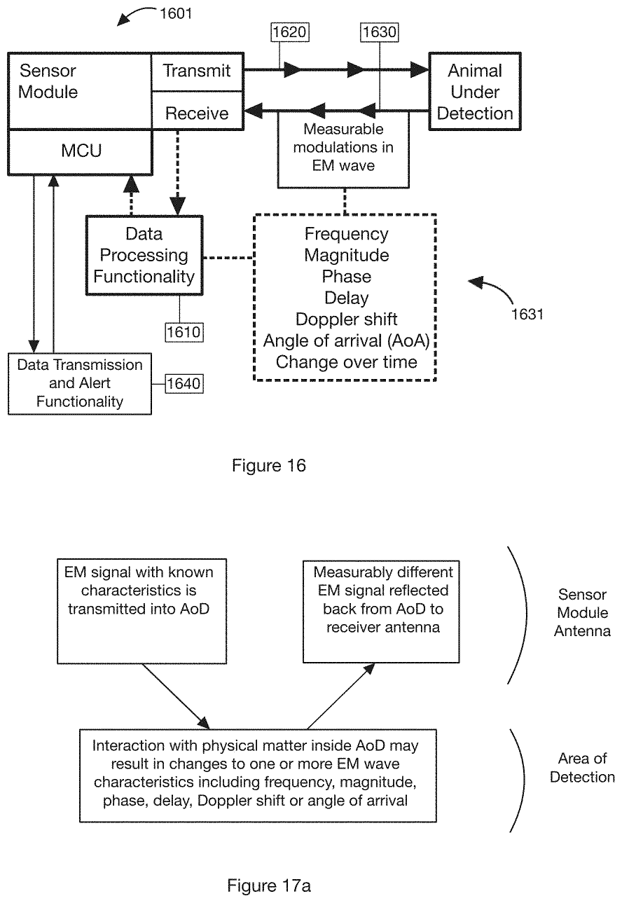

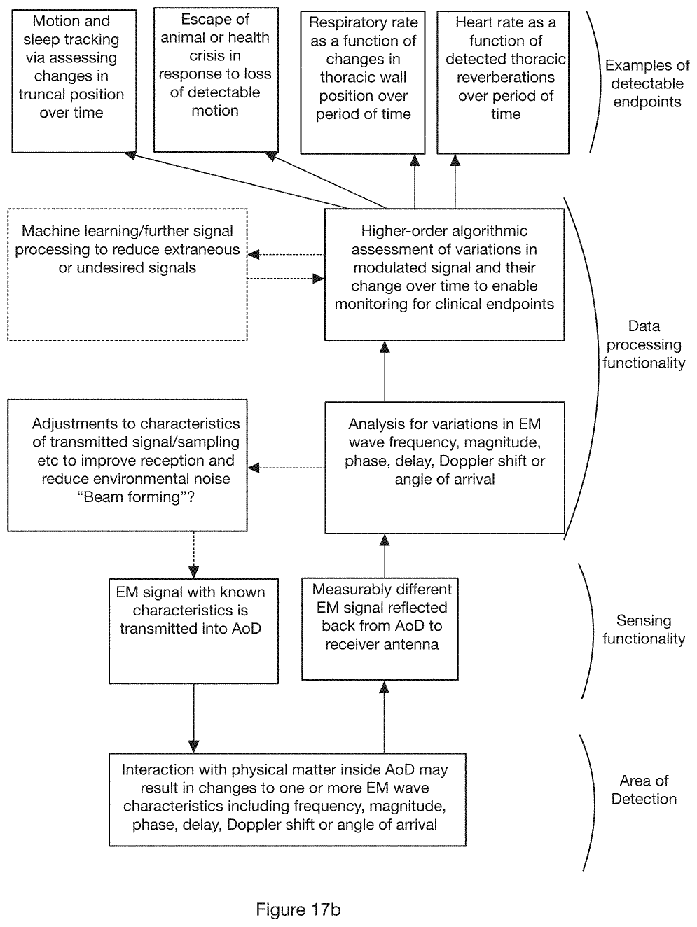

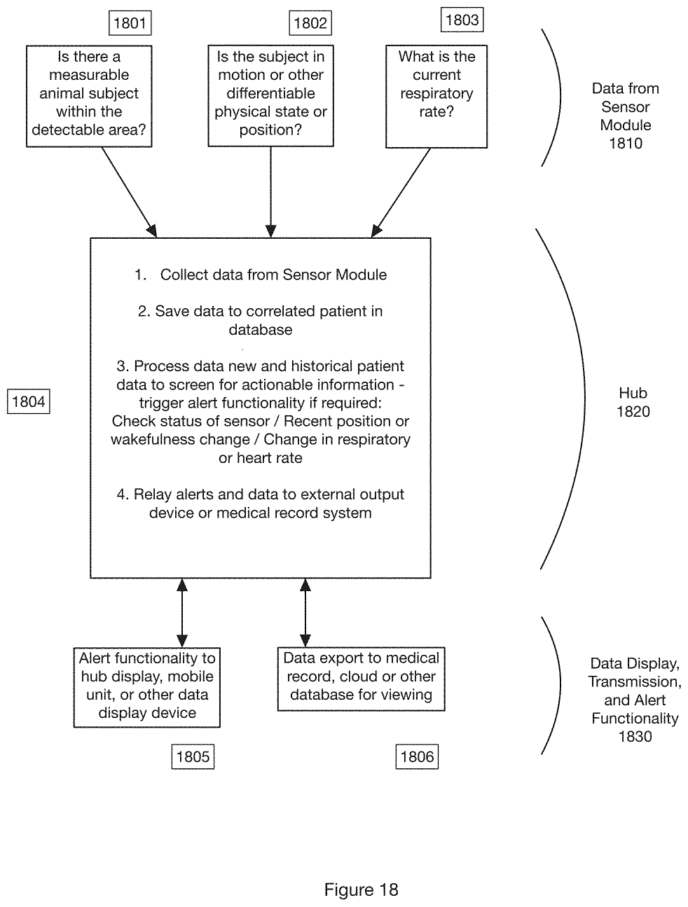

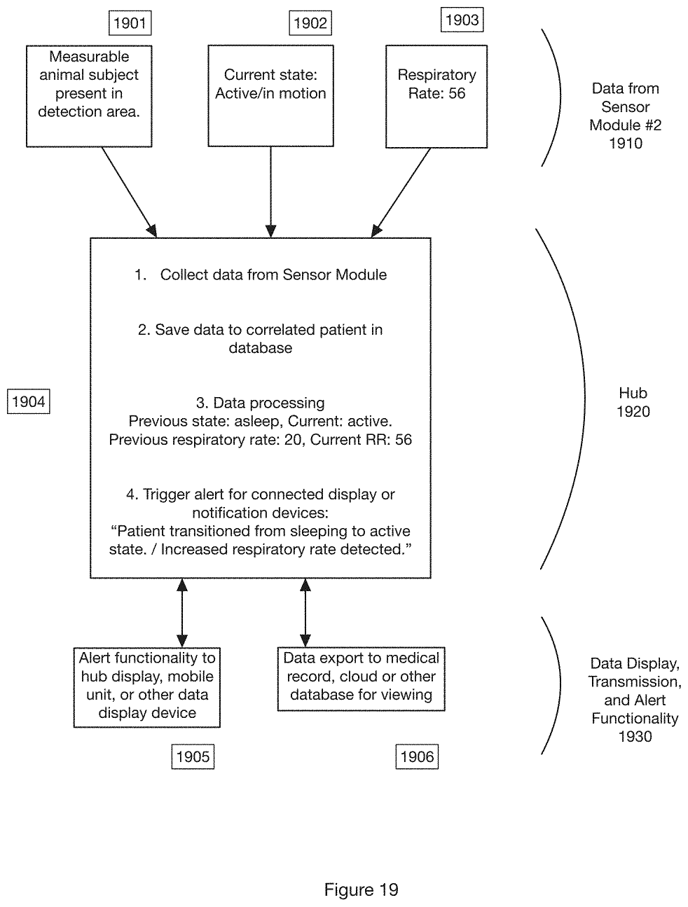

FIG. 2 shows a schematic diagram for one embodiment of the sensor module made up of microprocessor unit 240 & associated printed circuit board componentry 250. Also pictured is a transceiver 260, EM wave transmit antenna 270 and receive antenna 280 used in data sampling and collection further detailed in FIG. 16. The sensor module will include a connector or connectors to allow for data 210 and power 220 transfer to and/or from the unit. The unit may include a battery 230 as an auxiliary (supplemental) power source. Additionally, the sensor module could include a separate wireless transceiver and antenna for wireless communication of sampled data to the hub, cloud or mobile monitor similar to the wireless functionality described in the hub module 360, 361 and further referenced in FIG. 10. The sensor module will be housed in a case 290 made of any form of resin including plastic, bio-based, or other resin combination.

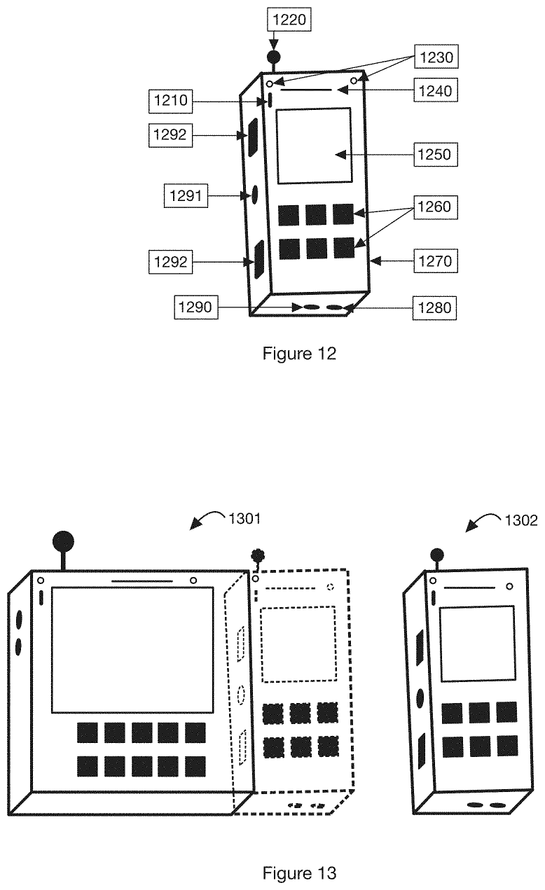

As shown in FIG. 3, the hub module made up of microprocessor unit 340 & associated printed circuit board componentry 350 to accept the data relayed from the sensor module(s) detailed in FIG. 2. Following data processing further outlined in FIG. 5, the data from one or more sensor modules will be sent to the end-use software housed on a computer, tablet, or smart phone. This data may be sent via a wired data connection 310 or wirelessly via a transceiver 360 and external antenna 361 over 3G/4G/5G/LTE, WiFi, or Bluetooth to an external computer, database, display, mobile unit, or other receiving device. The hub will include a connector or connectors to allow for transfer of power 320 to the hub module, to a sensor module(s), or for use with a docking/charging solution for a handheld monitoring unit as illustrated in FIGS. 12-13. The same or additional connections may be used to transfer power or data to local memory devices, or any other data transfer interface to as computers, routers, modems, or other. Alternative implementations of the hub module may include an axillary battery 330, audio output functionality 370, a visual display or indicator 371, or activation & control buttons 372. The hub module will be housed in a case 380 aluminum or other metals, plastic, bio-based or other resin combination.

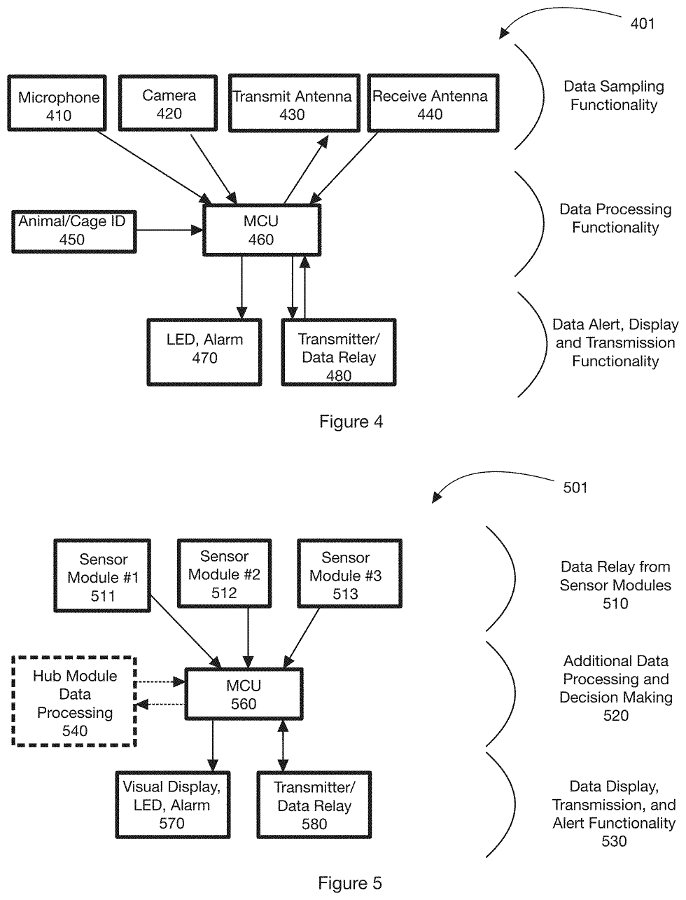

FIG. 4 demonstrates the relations between different functionalities of the sensor module 401. Electromagnetic waves are transmitted via antenna 430 and the attenuated EM waves from the monitored animal are then sampled via the receive antenna 440, a functionality which is further detailed in FIGS. 15-16. This information is relayed to the sensor module MCU 460, which can also receive data inputs from sensor 410 and sensor 420. Animal identification and other configuration information 450 can be relayed to the sensor module MCU via the hub, if needed. Once collected, data is processed by the sensor module MCU and information is then communicated via the hub module for further processing and transferred to the pointed database, app, or other end-use software or solution 480. The processed sensor module information can also be forwarded to the alert components 470 if necessary. Such alerts could be blinking or alternating lights (including colors) for a variety of notifications including power issue, connection issue or failure, or otherwise.

FIG. 5 gives an example of hub module 501 functionalities upon receiving data transmitted from individual sensor modules 511, 512, and 513. The hub module MCU 560 will accept raw or initially processed sensor module data 510 including EM sensors data, cameras, or other input devices and perform further algorithmic or other data processing functionalities 540.

The hub module MCU utilizes data processing 540 to analyze incoming sensor module data and will trigger alert 570 or data relay 580 functionalities as appropriate. Depending on configuration, the data be presented to the end-user via software housed on a computer, tablet, smart phone application, or within the hub itself. There will also exist the ability to communicate patient data from medical records software or other database back to the hub or sensor if needed (e.g. image of patient, name, age, weight, breed, medical record number, symptoms, diagnosis).

Referenced hub module data processing and decision making 520, 540 and data alert, display, & transmission functionality 530 are further detailed in FIGS. 18-19 and FIGS. 20-23 respectively.

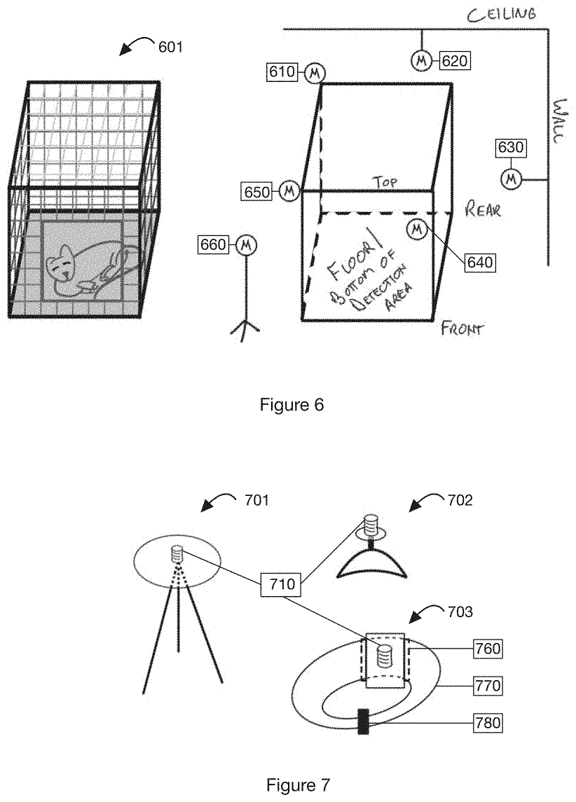

FIG. 6 shows some placement options for the sensor in such an enclosure. Additionally, the sensor may be deployed in a stand-alone function 660, 701 for viewing animals in various larger enclosure types or situations. The sensor module can be deployed with a specific animal or cage pairing or as a proximity monitor assigned to a specific area or region of the animal habitat or environment, such as mounted to the ceiling 620 or wall 630 in close proximity to a cage or inside an enclosure.

FIG. 7 illustrates three potential mounting solutions for the sensor module. A common connector 710 is used to secure the sensor module to a freestanding mount 701, an adhesive or suction attachment 702, or to incorporate with an tension mounting system 703 comprised of a mounting bracket 760, strap or cable 770, and an adjustable mechanism 780 to secure the sensor to a cage door, post, railing, tree, or other suitable sensor placement location.

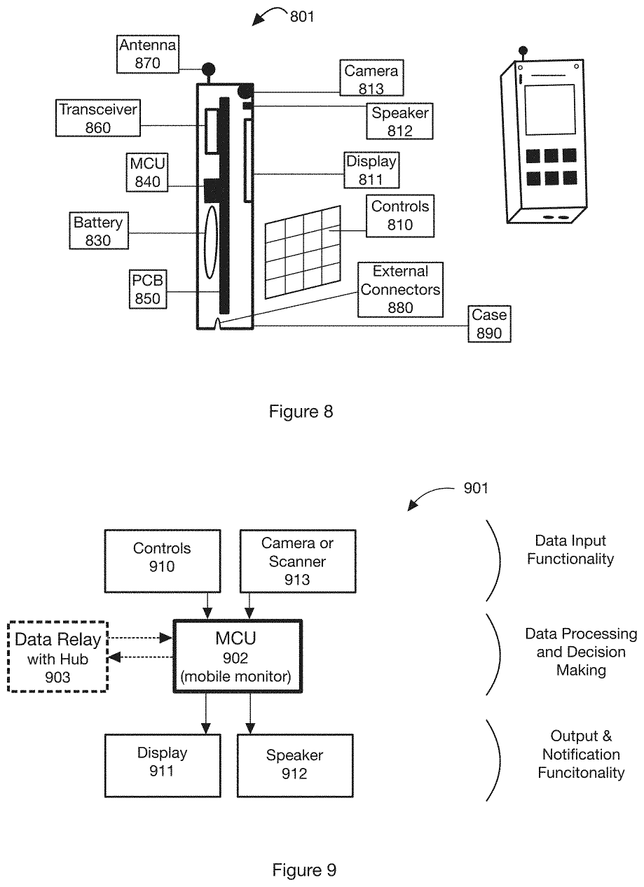

FIG. 8 shows a schematic diagram for a mobile monitor unit 801 made up of microprocessor unit 840 & associated printed circuit board componentry 850. Also pictured is a transceiver 860 and external or printed antenna 870 for wireless data relay with the hub or sensor module(s) referenced in FIG. 5. The MCU can receive input from activation and control buttons 810 or other integrated input devices such as a camera or optical scanner 813. Components for audio output 812 or display functionality 811 may also be included. There are external connector(s) 880 for data or power transfer. The mobile monitor unit will be housed in a case 890 made of aluminum or other metals, plastics, bio-based, or other resin combinations.

FIG. 9 is a block diagram relating some component functions of a mobile monitor unit 901, specifically focusing on where they differ from those of the hub unit. The mobile monitor MCU 902 receives data input relayed from the hub 903, as indicated in the previously referenced hub data relay functionality 580 in FIG. 5. The MCU can receive input from activation and control buttons 910 and attached or integrated input devices such as a camera or optical scanner 913 for the purposes of device configuration, notification response, or other system control. The MCU will process data received from device inputs and data relay from the hub and, when necessary, can output via attached audio 912 or display 911 components.

FIG. 10 is an external diagram of a possible embodiment of the sensor module in FIGS. 1-2. There are external ports to relay data 1010 and power 1020. An indicator light 1030 and a power or activation switch 1040 are also shown. An identification code or other linking system 1050 for sensor/hub configuration is also referenced. The bottom of the sensor module has a mounting connector 1060 for use with equipment as discussed in FIG. 7. A wireless data relay 1070 may provide the ability to wirelessly relay sensor module data to hub or other end-use solution wirelessly via 3G/4G/5G/LTE, Wi-Fi, or Bluetooth.

FIG. 11 is an external diagram of a possible embodiment of the hub module referenced in FIGS. 1, 3. There are external ports for connections to relay data 1180 and power 1190 as well as an external antenna 1120 for previously discussed wireless communication protocols. There is an activation switch 1110 and controls for user input 1160, if needed. Indictor lights 1130, a speaker 1140, or visual display 1150 may be used to communicate information to the user. Docking connectors 1192 and connection for data & power transfer 1191 to a detachable monitoring component are shown here and further detailed FIGS. 12-13.

FIG. 12 is an external diagram of a possible embodiment of a mobile monitor previously described in FIG. 8. The activation switch 1210, external antenna 1220, indicator lights 1230, speaker 1240, display 1250, input controls 1260, and data 1280 & power 1290 connectors are similar in purpose and function to those described in FIG. 11. The connectors 1292 and ancillary data & power connection 1291 are reciprocal components to the docking connectors 1192 and data & power connections 1191 referenced in FIG. 11 for use with an integrated docking/pairing solution between the hub and mobile monitor.

FIG. 13 shows an illustration of a possible embodiment of the hub module 1301 with a detachable mobile monitor unit 1302, with components similar in functionality and structure to those to each independently described unit in FIGS. 11-12. Other embodiments of the hub module may forego housing some or all display, alert, or configuration component within the hub itself and instead integrate excluded functions into the mobile monitor unit. Conversely any configuration, display, or alert functionality described as pertaining to the mobile monitor unit could be integrated into the hub module, either as a permanent component or via the described docking solution.

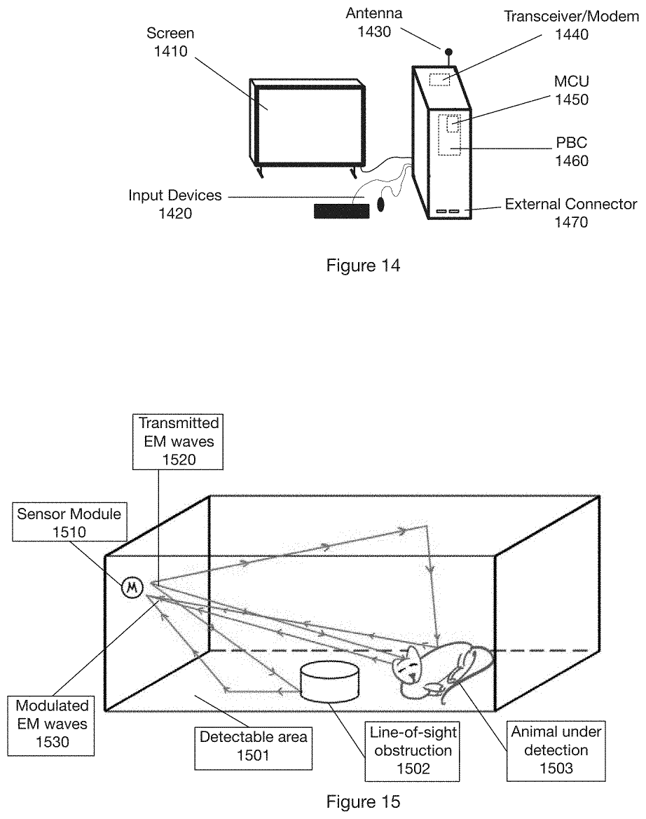

FIG. 14 diagrams a personal computing station comprised of the following components: screen 1410, mouse & keyboard for input 1420, antenna 1430, transceiver 1440, microcontroller unit 1450, printed circuit board or other ancillary circuitry 1460, and external connectors for data transfer 1470. The PC (personal computer) can run software to display sensor module data relayed from the hub unit or may directly perform some or all functions of the hub and mobile monitor previously described.

FIG. 15 is an overview illustration of data sampling using EM sensing of an animal subject, via the sensor module described in FIG. 2. The sensor module 1510 operates as the primary data collection instrument. Transmitted EM waves 1520 interact with any material substance inside the detectable area 1501. Upon interacting with matter inside the detectable area 1501, the modulated EM waves 1530 that have been reflected back to the sensor module are converted to a low-frequency signal which is distinctly different in character from the originally transmitted EM signal. As further detailed in FIG. 16, these variations in wave characteristics and their change over time can be used to build a physiologic profile of the animal subject.

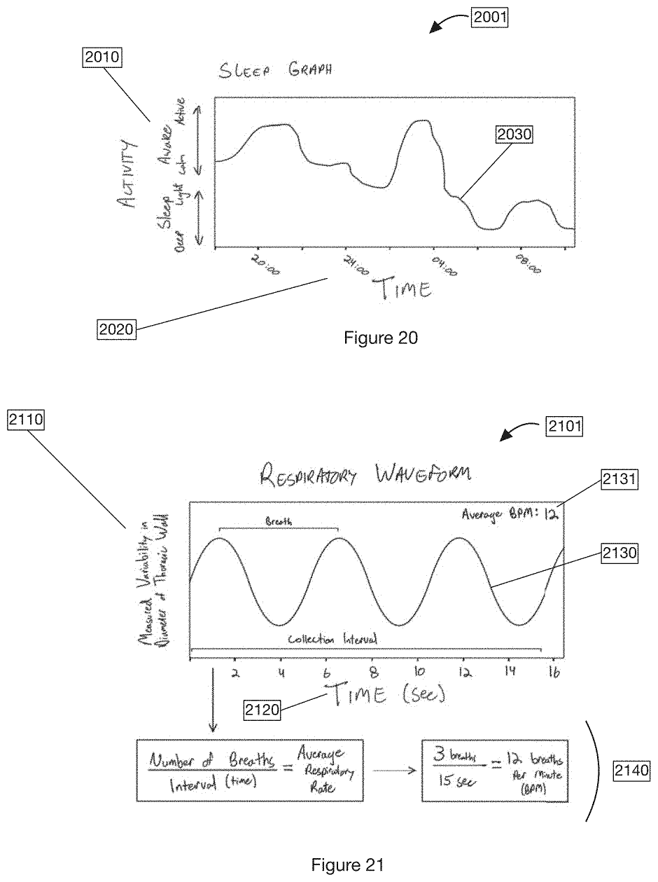

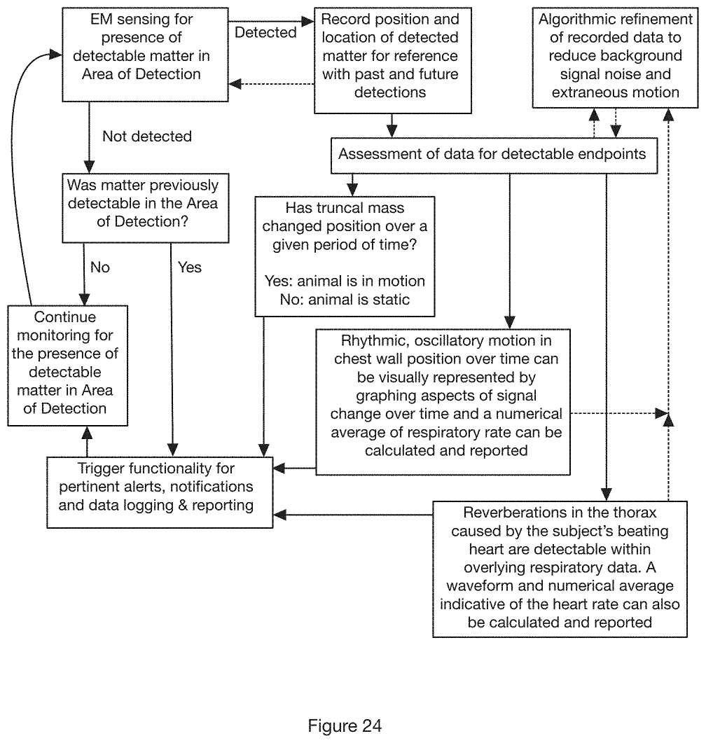

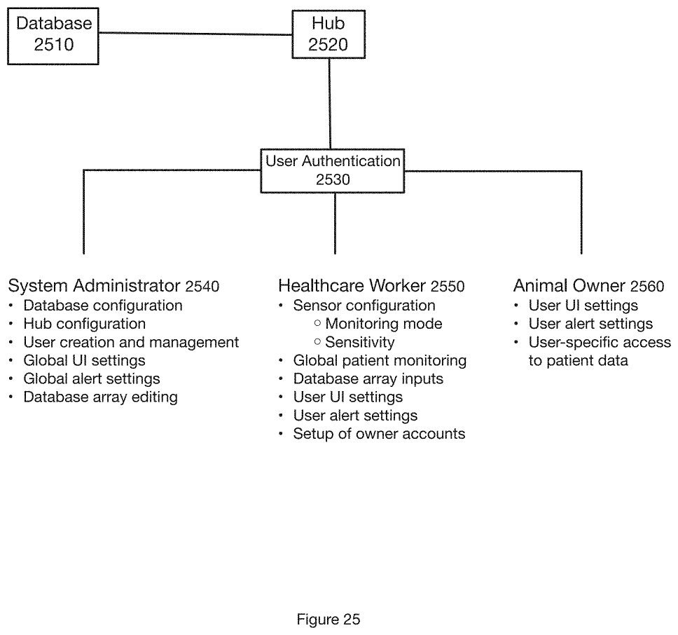

The block diagram in FIG. 16 further details a sensor module's use of EM sensing to gather physiologic data from an animal subject. The sensor module 1601 transmits EM waves 1620 while concurrently measuring modulations in received EM waves 1630 including, but not limited to, frequency, magnitude, phase, delay, Doppler shift, angle of arrival (AoA), and their changes over time 1631. The sensor module is able to adjust transmitted EM waves via beam-forming and other signal filtering and processing refinements to further improve data sampling 1610. By comparing changes in signal characteristics including frequency, magnitude, delay, doppler shift and angle of arrival, the sensor is able to minimize interference from patient motion and interactions from the environment & nearby animals or caregivers. The sensor module data is then relayed to the hub 1640 as also referenced in FIGS. 4-5. The methodology used in extracting vitals and other physiologic data from the converted EM wave signal is further outlined in FIGS. 20-24.

FIG. 18 shows one embodiment of decision making process of the hub with regards to received sensor module data for a patient. The hub module 1820 receives data from a specific sensor module 1810 paired with a patient/cage. Using measured variations in EM wave data, the hub software uses established algorithms and machine learning to interpret presence 1801, motion 1802, and respiratory 1803 data for the patient. Further refinement of data at the level of the hub enables for screening against thresholds with alert functionality to inform end-users if monitored values are outside of previously established ranges or if the measured value changes too quickly or too frequently in a given period of time 1804. Visual data outputs may be displayed on the hub or transmitted to other described end-user solutions and can incorporate audio or other alert functionality on the hub or end-use as device functionality permits 1805. The data is also made available for visual display or import into a medical records software or other database 1806.

FIG. 19 is a flow diagram for implementation in a patient with an anomalous increase in respiratory rate and activity change. Incoming data 1910 transmitted from sensor module #2 indicates that the associated patient is currently detected in the cage 1901, has a high degree of motion 1902, and has a respiratory rate of 56 breaths per minute 1903. Data from sensor module #2 is transmitted to the hub 1920. Shown in block 1904, the hub takes the collected sensor data and saves it to the corresponding patient in the database. Further data processing and historical comparison is used to determine any actionable changes in measured parameters. Alert and data display functionality 1905 is triggered to alert the end-user of the notable changes in sleep status and respiratory rate and the data is exported to the patient's medical record or other attached database 1906. The hub's transmission, display, and alert functionality 1930 referenced herein should be considered interchangeable with any end-user display solution or database including, but not limited to, a mobile monitor, integrated hub display, computer program, phone or tablet application, web interface, electronic medical record, or other connected database solution.

### Physical/Physiological Parameters Measurement

The sensor module utilizes wave-based wireless sensing to measure physical/physiological parameters such as, but not limited to, pulse/heart rate, respiratory rate, mobility, eating, drinking, or other types of behavior measurement, such as tail wagging, barking, ear and head movement, sleep, panting, pacing, trembling, emesis, collapse.

The data once viewed by end-users may then be analyzed & able to determine the animal's condition on the given variables. Further, the software application may provide alerts to the end-user for various criteria such as any time motion is detected (motion/static), degree of motion (high/medium/low/none), respiration status (numeric or otherwise quantified), heart rate levels (numeric or otherwise quantified) or a stop of all data collection (potentially indicating a catastrophic event such as death of the subject).

Measured differences of variables listed in box 1631 when comparing the transmitted and received (modulated) EM waves can be analyzed via pattern recognition and other algorithmic processing to correlate to specific physiologic processes of the animal under detection. Electromagnetic wave sensing can be used to measure respiratory rate & function by correlating the modulated signal's phase shift to the rate of expansion of the thoracic wall during breathing or by correlating the rate & character of cardiac muscle contraction to the patient's heart rate & function. Moreover, presence detection and movement data can be gathered by analyzing modulated wave characteristics such as delay, Doppler shift, angle of arrival and other variations in electromagnetic wave character.

FIG. 20 is an example illustration depicting one method of tracking and displaying activity & sleep data. Using EM wave sensing to plot the subject's measured activity level 2010 over a set period of time 2020, a line plot 2030 is able to convey the previous and current activity data to the end-user or solution. Additionally, data regarding a subject's distance from the sensor & level of motion or activity could be used to build a graphical representation of sleep history, degree of comfort or pain control, Sensor calibration could also be used to monitor the length of time a patient was in a specific postural position, a specific area, or to monitor how many times an entered or exited a specific space within the detectable area.

Moreover, FIG. 21 is an example depiction of respiratory rate as the monitored physiological indicator based on the phase shift recorded in the modulated EM wave. The graphed respiratory data 2101 shows how modulated EM data can be algorithmically processed to generate a waveform 2130 representing the distinct, cyclical expansion and compression of the thorax 2110 associated with respirations over a period of time 2120. A numerical average of the recorded respiratory rate 2131 can be obtained by measuring a set number of respiration cycles in a specific period of time 2140.

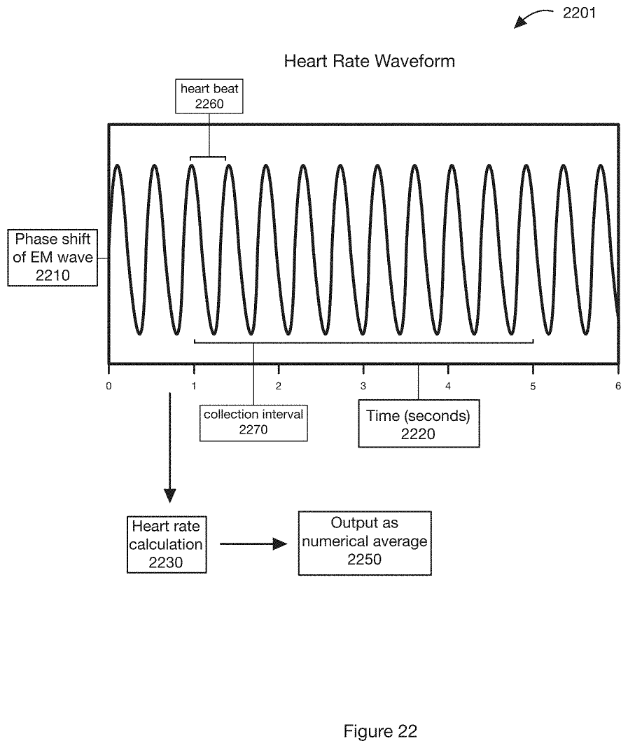

The waveform shown in FIG. 22 is generated by applying a similar algorithm to isolate and monitor for a phase shift between transmitted and received EM wave signals 2210 that can be correlated to the contractile “beat” of the cardiac musculature displayed over time 2220. Once a measurable shift in signal phase can be correlated to cardiac contraction, data processing 2230 is used to calculate a numerical average of heart rate as a measure of how many phase shifts correlated to the subjects heart beat 2260 are detected in a selected interval of time 2270.

To achieve this level of accuracy, signal filtering and noise reduction techniques are applied to the same collected EM data used in previously described presence, motion, and respiratory monitoring and is further analyzed for changes in signal frequency, magnitude, phase, delay, Doppler shift, and Angle of arrival while incorporating analysis of other physiologic parameters into the assessment of cardiac function or other “higher level” issue (needs rewording).

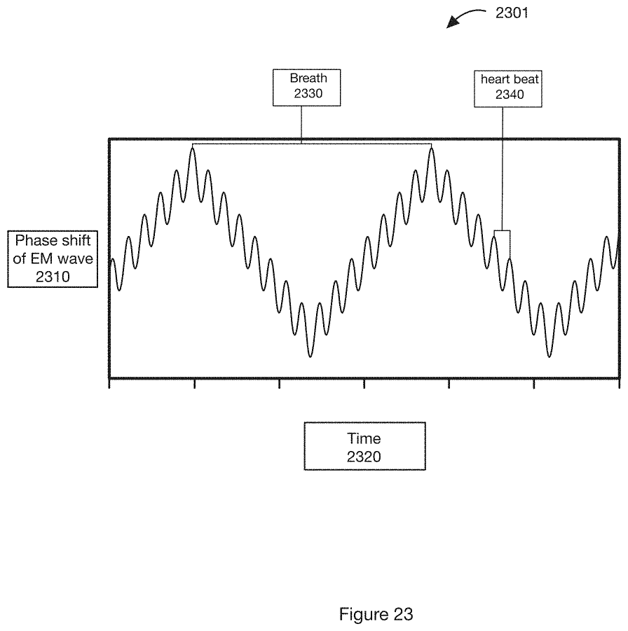

Combining the signal filtering processes previously described in FIG. 16 and applying additional algorithmic analysis is used to further extract information from the modulated EM wave date. For example, utilizing a signal's phase shift to monitor cardiac rate in a subject preferably uses algorithmic filtering of any phase shift attributable to respiratory rate—as in, a more discernible cyclical phase shift used in assessing respiratory rate can itself mask a much smaller phase shift attributable to the subject's cardiac rate. FIG. 23 shows an example graph of phase shift 2310 in relation to time 2320 which cyclical phase variations from respiratory rate and heart rate are simultaneously discernible within the same data set. The graphed data 2301 represents the collected phase shift readings after sensor and initial hub level processing have minimized signal interference from subject motion, the ambient environment or nearby animals & caregivers. As illustrated, the larger-magnitude phase shifts 2330 correlating to the patients breathing rate are algorithmically differentiated from the lesser-magnitude phase shifts 2340 result from the detected EM wave phase shift secondary to cardiac contraction.

Alternatively, factoring measured data such as respiratory rate, heart rate, and motion into logic trees can be used to help differentiate between an overly excited animal from one that is having a health crisis such as a seizure or an animal that is resting calming from one that loses consciousness or experiences respiratory or circulatory collapse.

The app or interface utilized via tablet, computer, local device or otherwise may take multiple formats of user interface. In one format, a user may have a login credential to point them to a specific animal for observation. In another, the user or veterinary professional may have access to the full array of devices setup. This array would be visually represented in a module format allowing for reconfiguration based on individual preference or priority. Additionally, administration features, settings, and pre-configured alerts may be utilized in the landing home page. Once an option has been selected, the app will point to the designated area such as the specific cage. The visual output at this point may provide the animal name, a photo or live image if a camera is employed, vital rates, and associated parameters or alert systems setup for this subject.

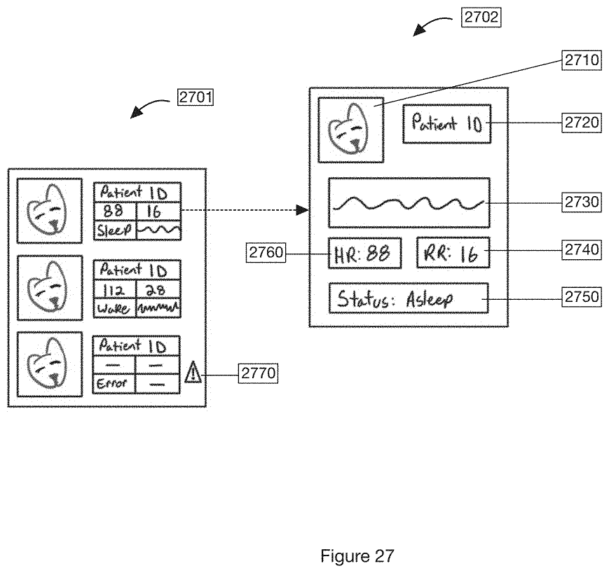

The end user may visually view the software application via a multitude of devices such as computers, laptops, smart-phones, tablets, or others. The application will allow for unique login by account holders with various permissions specific to the user or account type authenticated 2530. A system administrator account 2540 will be enabled to create and manage accounts for staff or other healthcare workers and perform higher-level configurations of the database, hub, and other end-use solutions. Healthcare worker accounts 2550 will be enabled to configure sensor and other patient data. Furthermore, the application will allow for the creation of external accounts for pet owners 2560 to be provided QR codes, tokens, logins, or other credentials to access a specific animal's data in real time. As further referenced in FIG. 27, the software will provide a visual output of the animal (possibly a photo if uploaded or inputted), name, animal type, location of the sensor (i.e. Cage 3, cage 4, lab 1, etc.), respiratory & heart rate in various embodiments (raw data, wave-form, average date or any output permutation possible), and any threshold selections for alerts. Alert settings may be as specific as certain levels of breathing, heart rate, or movement, or even as simple as “movement/no movement” alerts. The application will also have a database function to aggregate data which may be stored locally on a computer or hard drive device designated the main area. This data may also be directed to a cloud or remote solution for aggregation and recall at a later time. The algorithm will continue utilizing this aggregated data to improve the accuracy of the sensor and interpretation of data via machine learning & AI methods.

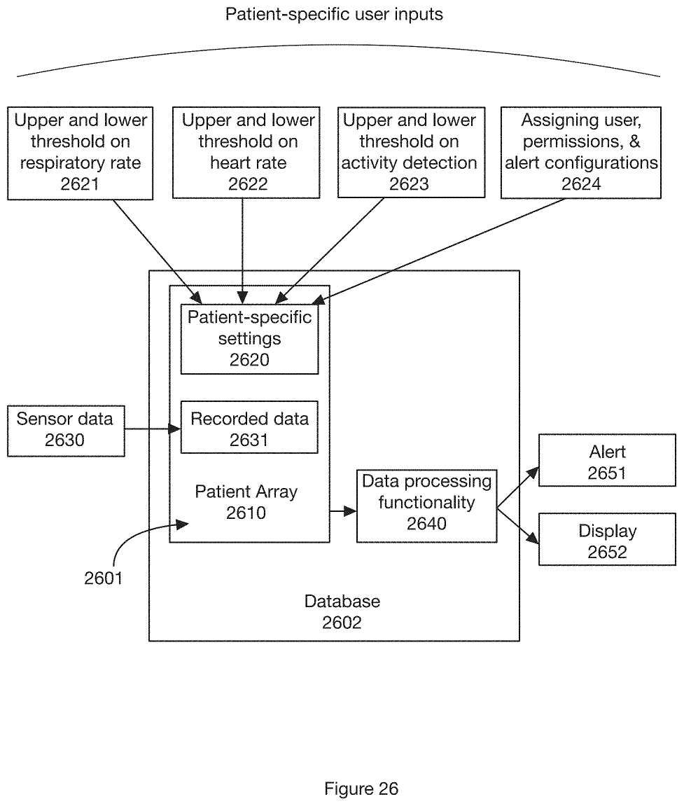

FIG. 26 demonstrates a potential process flow for the application and method by which the user can interface. Information regarding the patient 2601 is stored in a database 2602 within the specific patient array 2610. Parameters for upper & lower respiratory rate 2621, upper & lower cardiac rate 2622, specific motion indicators or frequency 2623, or other configuration variables 2624 can be entered into as patient-specific settings 2620 within the appropriate patient array. Sensor data 2630 from the sensor module linked to a patient is entered as recorded data 2631 in the corresponding patient array. This data may be aggregated in any database by which the user has designated, either on the local device or pointed to a cloud or separate storage unit. Data processing functionality 2640 may be deployed to screen for deterioration in health status or in response to parameters exiting the patient-specific thresholds and to trigger previously described alert 2651 and display functionality 2652 where applicable.

FIG. 27 shows an end-user UI implementation to display relevant patient data. Illustration 2701 demonstrates how health data from multiple patients can be displayed simultaneously with an alert 2770 notifying of a change or issue with a specific patient. Illustration 2702 labels specific patient data that may be displayed including an image or live feed of the patient 2710, patient identification & medical information 2720, a waveform or other graphical depiction of measured data 2730, numerical averages of the respiratory rate 2740 & heart rate 2760 or any other measured or otherwise relevant notifications or data. Binary measures, such as sleep state 2750, position of animal, presence of motion, or transition into an urgent or otherwise predetermined state may be displayed visually with the option of audio cues or push notification.

### Summary of Operation

The data collected wirelessly will avoid stressing the subject animal as well as provide continuous monitoring of any variety of situations. Such indications could provide distress when an animal unprovoked begins moving in extreme manners. Other indications could be if an animal experiences a fatal or otherwise urgent event and all movement & breathing stops, alerting professionals and providing critical moments for resuscitation and other intervention.

Measured parameters are relayed (via wired or wireless connection) to a hub, which acts as a centralized controller for information relay to other devices such as, but not limited to, a computer, smartphone app, electronic medical record, or local or cloud-based storage solution via USB, serial, ethernet, or some other transfer method.

A camera or other sensors may be utilized either in conjunction with or directly as part of the overall assembly of the sensor to further provide visual data of the animal and provide additional details, variables, and interpretation to the software to further improve accuracy and functionality. Such information can provide important details as to degree of motion as well as which part of the animal may be moving.

What is claimed is:

1. A system for wirelessly, non-invasively, and without physical contact monitoring the physiological data of non-human animals comprising:
a module deploying an EM (electromagnetic) sensor for wirelessly transmitting electromagnetic waves to an area in the vicinity of an animal, the sensor module receiving modulated signals back from the animal that have been modulated as a function of physiological characteristics of the animal, and the sensor module interpreting the modulated signals and providing information about a physiological characteristic of the animal to a user.

2. The system of claim 1 wherein the EM sensor is configured to transmit electromagnetic waves in the mmWave range to an area in the vicinity of the animal.

3. The system of claim 1 wherein the EM sensor is configured to transmit electromagnetic waves in the 24 GHz to 28 GHz range to an area in the vicinity of the animal.

4. The system of claim 1 wherein the EM sensor is configured to transmit electromagnetic waves into an animal enclosure.

5. The system of claim 1 wherein the EM sensor is configured to transmit electromagnetic waves to a non-human subject within a 3 meter radius of the EM sensor.

6. The system of claim 1 which further comprises:
a visual interface by which a veterinary professional may extract waveform and motion data & interpret this as respiratory and heart rates combined with motion and occupancy sensing to determine factors attributable to the well-being the of the animal, including stress levels, activity levels, respiratory or hearts rates, or other factors attributable to changes in the animal's health, including motion events, lack of motion, loss of containment or fluctuations or deviations in respiratory rates or heart rate.

7. The system of claim 1 which further comprises:
an alerting device configured to alert or otherwise notify an end user, veterinary professional, or other consumer or pet owner of changes in animal status, such as variations in heart rate, respiratory rate, motion, lack of cage occupancy.

8. The system of claim 1 which further comprises:
a visual interface by which a consumer or pet owner may access live pet data to remotely monitor their pet or animal's condition while in a remote location.

9. The system of claim 1 wherein the visual interface is configured for a user to customize and select alert criteria.

10. The system of claim 1 which further comprises:
a processor which aggregates data over time to determine anomalies or establish new baselines in animal behavior including respiratory rate, heart rate, and/or motion via various sensor or visual methods applying machine learning to identify patterns and alert professionals to unique or outlying events and resulting in identification of variations or deviations from typical or expected behavior or health.

11. The system of claim 1 which further comprises:
a plurality of EM sensors, a camera and/or microphone either: inside, outside, or integrated into an animal enclosure to report back meaningful data to end users.

12. The system of claim 1 wherein the plurality of EM sensors, a camera and/or microphone is integrated into existing cage or enclosure architectures.

13. A system for noninvasively monitoring physiological data of non-human animals, said system comprising:
a sensor module having a transmit antenna, a receive antenna and a microprocessor,
the transmit antenna being configured to wirelessly transmit electromagnetic waves to an area in the vicinity of an animal,
the receive antenna being configured to receive modulated signals back from the animal that have been modulated as a function of physiological characteristics of the animal;
a hub having a processor and configured to receive signals from the sensor module; and
the processor in the hub being further configured to compare the signals from the sensor module with previously obtained data and generate an alert to a user if the comparison indicates an abnormal physiological characteristic of the animal.

14. The system of claim 13 wherein the sensor module is configured to detect motion of the animal and trigger collection of electromagnetic signals transmitted back from the animal; and
the hub uses a phase shift of the electromagnetic signals transmitted from the sensor module to determine a physiological characteristic of the animal in motion.

15. The system of claim 14 wherein the physiological characteristic is selected from the group of respiratory and heart rate conditions.

16. The system of claim 14 further including a camera or a microphone for sensing information about the animal in a cage, with the sensor module transmitting visual or sound information to the hub.

17. The system of claim 13 which further comprises:
a mobile monitoring unit communicating with the hub, the mobile monitoring unit having control buttons configured to transmit information to the hub.

18. The system of claim 13 wherein a plurality of sensor modules are located in a veterinary office having a plurality of cages, each sensor module being located adjacent a given cage.

19. The system of claim 13 wherein the sensor module is configured to sense pulse/heart rate, respiratory rate, mobility, eating, drinking, tail wagging, barking, ear and head movement, sleep, panting, pacing, trembling, emesis, or collapse.

20. The system of claim 13 which further comprises:
A visual interface by which a consumer or pet owner may access live pet data via a system access to remotely monitor their pet or animal's condition while in a remote location.
